(19) United States
(12) Patent Application Publication
(10) Pub. No.: US 2014/0377295 A1
(43) Pub. Date: Dec. 25, 2014

(54) INFLUENZA VACCINES CONTAINING MODIFIED ADENOVIRUS VECTORS

(71) Applicants: Hildegund C. Ertl, Philadelphia, PA (US); Dongming Zhou, Philadelphia, PA (US)

(72) Inventors: Hildegund C. Ertl, Philadelphia, PA (US); Dongming Zhou, Philadelphia, PA (US)

(73) Assignee: THE WISTAR INSTITUTE OF ANATOMY AND BIOLOGY, Philadelphia, PA (US)

(21) Appl. No.: 14/119,150

(22) PCT Filed: May 23, 2012
(86) PCT No.: PCT/US2012/039051

§ 371 Date: Aug. 14, 2014

Related U.S. Application Data
(60) Provisional application No. 61/488,904, filed on May 23, 2011.

(51) Int. Cl. C07K 14/005; C12N 7/00

(52) U.S. Cl. CPC C07K 14/005; C12N 7/00; C07K 2319/40; C12N 2710/10041; C12N 2760/16134; C12N 2760/16141; A61K 39/00
USPC 424/186.1; 530/350; 530/358; 536/23.4; 435/320.1

(57) ABSTRACT

This disclosure provides universal influenza vaccines which can provide extended protection for several years, provide improved protection to circulating influenza strains that were not predicted accurately for annual vaccine manufacturing, and provide protection against newly emerging strains of influenza virus which carry the potential for establishing global pandemics.

This application claims the benefit and incorporates by reference Ser. No. 61/488,904 filed on May 23, 2011.

This application incorporates by reference the contents of an 8.67 kb text file created on May 21, 2012 and named “00048600038sequencelisting.txt,” which is the sequence listing for this application.

Each reference cited in this disclosure is incorporated herein by reference in its entirety.

### BACKGROUND OF THE INVENTION

Influenza A viruses cause severe illness in 3-5 million people worldwide and are linked to 250,000-500,000 deaths each year. Two prototypes of influenza vaccines, trivalent inactivated vaccines (TIV) for intramuscular injection and attenuated virus (e.g., FLUMIST®) for intranasal application are available for vaccination of children and adults up to age 49. Only TIVs are approved for individuals 50 years of age and older. Although highly recommended for vulnerable population such as children and the elderly, influenza vaccines only provide limited protection, as shown by statistical analyses of vaccine trials. The viral surface proteins, which are the targets of neutralizing antibodies and the main correlates of vaccine-induced protection, mutate rapidly, and mismatches between the predominantly circulating strains and the vaccine component can further reduce vaccine efficacy. Re-assortment of genes from different strains can cause the emergence of new viral strains, which in turn, if they achieve sustained human-to-human transmission, can result in global pandemics. Such pandemics, in the extreme, can cause the death of millions of humans. Genetic modifications of influenza virus or selection of re-assortment viruses is technically feasible allowing for the development of potentially highly virulent viruses for use as bioweapons. Once a new influenza virus has been isolated and characterized, vaccines based on licensed prototypes can be developed within about 6-8 months. As a new strain of influenza virus can spread from a localized outbreak to every continent within less than 3 months, this delay in the case of a pandemic with a new highly virulent influenza virus may cost millions of lives.

There is, therefore, a continuing need for universal influenza vaccines that can provide baseline protection against a wide array of influenza viruses, including newly emerging strains.

### BRIEF DESCRIPTION OF THE DRAWINGS

FIG. 1. Graph showing the levels of antibodies against M2e two weeks after prime vaccination. C57BL/6 mice (n=10, 6-8 weeks old) were prime-immunized with 10¹⁰ vps (virus particles) of AdC68-3M2eNP (referred to in this figure and in Ser. No. 61/488,904 as “AdC68M2e(3)NP”). Combined with a heterologous boost vector, this regimen provides 80-90% survival upon challenge with 10 LD₅₀ of Influenza A/PR/8. The same dose of capsid-modified vectors with and without transgene were injected in parallel and resulted in significantly higher antibody responses.

FIG. 2. Construction of hexon-modified vectors. The flowchart shows the cloning of hexon R1- or R4-modified E1-deleted AdC68 vectors. The upper part of the figure shows the entire sequence of the E1-deleted molecular clone of AdC68, including the Mlu I sites that were used to excise the gene encoding the hexon. The lower part of the figure shows, from left to right, the pcDNA3.1 clone containing the viral hexon including the sites used for insertion of the M2e sequence into R1 or R4; the insertion sites for M2e; and the hexon-modified molecular clone.

FIG. 3. Protein was isolated from HEK 293 cells infected with AdC68 vectors carrying native (AdC68-rab.gp), R1-[AdC68-HxM2eS(R1)] or R4-[AdC68-HxM2eS(R4)] modified hexon under non-reducing conditions and analyzed by western blot with a monoclonal antibody to hexon. A monoclonal antibody to β-actin was used as a loading control.

FIGS. 4A-C. Expression of M2e. HeLa cells were infected with 10² or 10³ vp of vectors per cell. Twenty-four hours later, cells were stained with the M2e antibody (grey dots) or a negative control antibody (black dots), followed by staining with a PE-labeled secondary antibody and flow cytometry. The histograms show the levels of M2e expression over the numbers of events. FIG. 4A, AdC68-HxM2eS(R1); FIG. 4B, AdC68-HxM2eS(R4); FIG. 4C, AdC68-rab.gp.

FIG. 4D. Cells were infected with different amounts of vectors expressing the 3M2eNP fusion protein as a transgene product and analyzed for expression of the fusion protein by western blot with the monoclonal antibody to M2e. An antibody to B-actin was used as a loading control.

FIG. 4E. Plates were coated with purified AdC68 vectors carrying native hexon or hexon carrying M2e within R1 or R4. Plates were blocked, and treated with a monoclonal antibody to M2e, followed by incubation with an alkaline phosphatase-conjugated antibody and the substrate. Color changes were measured in an ELISA reader. The graph shows mean adsorbance (±SD) of substrate in wells that received different dilutions of the monoclonal antibody to M2e.

FIG. 5. Mice were immunized with vectors carrying M2e within R1 or R4 of hexon or with a vector with native hexon (AdC68-rab.gp). Sera were tested for neutralization of an AdC68 vector with naïve hexon expressing enhanced green fluorescent protein (EGFP). The graph shows the reciprocal neutralization titers of AdC68 with native hexon by antibodies induced with hexon-modified vectors.

FIGS. 6A-B show humoral responses to M2e. FIG. 6A, An M2e peptide ELISA was used to measure M2e-specific antibody titers in sera of ICR mice (n=10). Sera were harvested 5 weeks after priming (black bars) or 5 weeks after the boost (white bars). FIG. 6B, A cellular ELISA was used to measure antibodies from vaccinated C57Bl/6 mice (n=5). Sera were harvested 2 weeks after priming. Graphs show average titers±SD normalized towards a monoclonal M2e-specific antibody. *P<0.05.

FIG. 7. Frequencies of NP-specific CD8⁺ T cells in blood were assessed 5 weeks after priming or 5 weeks after boosting by tetramer staining. Graph shows mean frequencies of NP-specific CD8⁺ T cells of individual mice±SD. *P<0.05.

FIGS. 8A-D show the results of experiments testing protection against A/PR8/34 challenge. C57Bl/6 mice (n=5, FIG. 8A and FIG. 8B) or ICR mice (n=10, FIG. 8C and FIG. 8D) were immunized twice with different vectors. Mice that were primed with AdC68-3M2eNP were boosted with AdC6-3M2eNP. Control mice immunized with AdC68-rab.gp were boosted with AdC6-rab.gp. The other groups of mice were boosted with the same vector used for priming. Mice were challenged with 10LD₅₀ of A/PR8/34 virus 2 months after boosting. FIG. 8A and FIG. 8C, graphs showing mean weight loss after challenge. FIG. 8B and FIG. 8D, graphs showing survival after challenge.

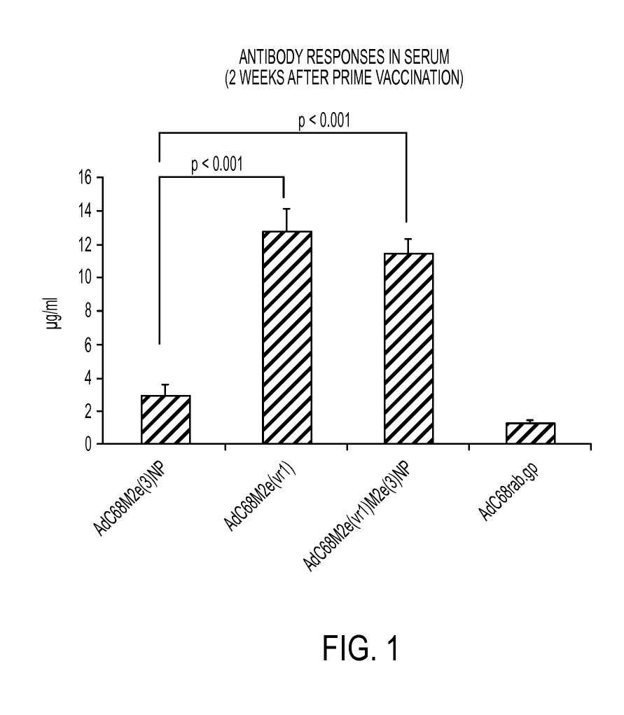

### DETAILED DESCRIPTION

This disclosure describes potent, universal vaccines against influenza that offer the advantage of replacing an annual influenza vaccine with vaccines that can provide extended protection for several years. In addition, the disclosed vaccines may provide improved protection to circulating influenza strains that were not predicted accurately for annual vaccine manufacturing. The disclosed universal influenza vaccines also can provide protection against newly emerging strains of influenza virus which carry the potential for establishing global pandemics.

The disclosed vaccine compositions are based on adenovirus (Ad) vectors that are derived from a chimpanzee, such as AdC68 (also called Sad-V25). Pre-existing neutralizing antibodies to these viruses are only rarely found at low titers in humans. The disclosed AdC vectors express a linear and conserved B cell epitope, preferably a matrix protein ectodomain epitope (M2e), on an accessible loop of the major coat protein of the vector particle, i.e., the hexon, which is present at 740 copies on the surface of an AdC virus. B cells are best induced by antigens that are expressed repeatedly and in an ordered fashion on a particle. Antigens arrayed in this fashion cross-link the B cell receptor, which initiates B cell activation and can drive a potent antibody response. Soluble antigen may activate B cells, nevertheless it is assumed that this requires higher concentration of antigens and that resulting antibody responses may be of lower quality regarding their specificity and binding strength.

M2e can be derived from any influenza A strain including, but not limited to, H1N1 (e.g., A/Puerto Rico/8/1934; A/Fort Monmouth/1/1947), H5N1 (e.g., A/Hong Kong/483/1997), H7N2 (e.g., A/Duck/Tasmania/277/2007), H1N2 (e.g., A/Swine/Korea/CY02/02), H2N2 (e.g., A/Leningrad/134/17/57), and H3N2 (e.g., A/New York/392/2004).

In some embodiments, the M2e is inserted in the hexon protein. In some embodiments, M2e is inserted in hypervariable region 1 (R1) of a hexon protein. In some embodiments, amino acids 142-144 of the hexon protein shown in SEQ ID NO:6 are deleted and M2e is inserted in their place. In some embodiments, M2e is inserted in hypervariable region 4 (R4) of a hexon protein. In some embodiments, M2e is inserted between amino acids 253 and 254 of the hexon protein shown in SEQ ID NO:6. Standard recombinant DNA methods can be used to achieve a deletion in the coding sequence for the hexon protein and to insert in place of the deletion a coding sequence for the M2e. See the working examples, below.

Because the AdC capsid is only accessible for a comparatively short time until virus has entered cells where the hexon is degraded, in some embodiments, Ad vectors in addition encode a fusion protein comprising additional M2e epitopes, preferably derived from up to three different influenza A virus strains, expressed in tandem from an expression cassette placed into the deleted E1 domain of the AdC vectors. Expression of M2e antigens from the expression cassette may be useful to extend the B cell response. The level of protection against influenza A infection can be increased by concomitant activation of CD8⁺ T cells to a conserved protein of influenza virus such as the adenovirus nucleoprotein (NP). Therefore, in some embodiments, the AdC vector also encodes NP, expressed as a fusion protein linked to the M2e epitopes. Nucleic acid molecules (either ribonucleic acid or deoxyribonucleic acid) encoding the fusion proteins can be constructed using standard recombinant nucleic acid techniques, e.g., as described in the working examples, below.

In some embodiments, fusion proteins comprise (1) a first matrix protein ectodomain from a first strain of influenza A virus (M2e₁); and (2) a nucleoprotein (NP) from a second strain of influenza A virus. In some embodiments, fusion proteins further comprise a second matrix protein ectodomain from a second strain of influenza A virus (M2e₂). In embodiments, comprising two matrix protein ectodomains and a nucleoprotein, at least two of these three components are from different strains of influenza A virus. In other embodiments, all three components are from different strains of influenza A virus. The two (or three) components can be in any order. In some embodiments, the M2e components of the fusion protein are derived from the same strain of influenza A virus as the M2e inserted into the hexon protein. In some embodiments, the M2e components of the fusion protein are derived from a different strain of influenza A virus than the M2e inserted into the hexon protein.

In some embodiments, fusion proteins comprise four components derived from at least two different influenza A strains: (1) a first matrix protein ectodomain from a first strain of influenza A virus (M2e₁); (2) a second matrix protein ectodomain from a second strain of influenza A virus (M2e₂); (3) a third matrix protein ectodomain from a third strain of influenza A virus (M2e₃); and (4) a nucleoprotein (NP) from a fourth strain of influenza A virus.

Suitable influenza A strains from which components of the fusion protein can be derived include H1N1 (e.g., A/Puerto Rico/8/1934; A/Fort Monmouth/1/1947), H5N1 (e.g., A/Hong Kong/483/1997), H7N2 (e.g., A/Duck/Tasmania/277/2007), H1N2 (e.g., A/Swine/Korea/CY02/02), H2N2 (e.g., A/Leningrad/134/17/57), and H3N2 (e.g., A/New York/392/2004).

In some embodiments, the first strain is an H1N1 strain. In some of these embodiments, the H1N1 strain is A/Fort Monmouth/1/1947. In other embodiments, the H1N1 strain is A/Puerto Rico/8/1934.

In some embodiments, the first strain is an H5N1 strain. In some of these embodiments, the H5N1 strain is A/Hong Kong/483/1997.

In some embodiments, the first strain is an H7N2 strain. In some of these embodiments, the H7N2 strain is A/Duck/Tasmania/277/2007.

In some embodiments, the fourth strain is an H1N1 strain. In some embodiments, both the first and the fourth strains are H1N1 strains and can be the same or different. In some of these embodiments, the first H1N1 strain is A/Fort Monmouth/1/1947. In other embodiments, the first H1N1 strain is A/Puerto Rico/8/1934.

In some embodiments, the four components are ordered, from N to C terminus, M2e₁-M2e₂-M2e₃-NP. In some embodiments, the NP and the M2e₁ are from A/Puerto Rico/8/1934; M2e₂ is from A/Hong Kong/483/1997; and the M2e₃ is from A/Duck/Tasmania/277/2007. In other embodiments, M2e components from three strains are in the order H1N1-H5N1-H7N2.

In any of the embodiments, the M2e inserted in the capsid can be derived from the same strain as the first strain (e.g., H1N1, H7N2, H5N1, or H7N2), the second strain, or the third strain, or can be obtained from a fourth strain.

In other embodiments, Ad vectors encode the fusion protein but do not comprise a modified hexon protein.

Production, purification and quality control procedures for Ad vectors are well established (Tatsis & Ertl, Mol Ther 10: 616-29, 2004). Ad vectors induce innate immune responses ameliorating the need for addition of adjuvants. They also induce very potent B and CD8⁺ T cell responses, which, due to low-level persistence of the vectors, are remarkably sustained (Tatsis et al., Blood 110: 1916-23, 2007). Pre-existing neutralizing antibodies to common human serotypes of Ad viruses such as serotype 5, which impact vaccine efficacy, can readily be avoided by the use of by serotypes from other species such as chimpanzees, which typically neither circulate in humans nor cross-react with human serotypes (Xiang et al., Emerg Infect Dis 12: 1596-99, 2006). In cases where prime-boost regimens are needed to achieve immune responses of sufficient potency, vectors based on distinct Ad serotypes are available (Tatsis & Ertl, 2004). Ad viruses and Ad vectors have been used extensively in the clinic where they were well tolerated. They can be applied through a variety of routes including mucosal routes such as the airways (Xiang et al., J Virol 77: 10780-89, 2003) or even orally upon encapsidation as was shown with vaccine to Ad viruses 4 and 7 used by the US military (Lyons et al., Vaccine 26: 2890-98, 2008).

Immunogenic compositions can be formulated using standard techniques and can comprise, in addition to the adenovirus vectors described above, a pharmaceutically acceptable vehicle, such as phosphate-buffered saline (PBS) or other buffers, as well as other components such as antibacterial and antifungal agents, isotonic and absorption delaying agents, adjuvants, and the like. In some embodiments, the compositions are vaccine compositions and can be administered in combination with one or more other vaccines, including other influenza vaccines (e.g., seasonal vaccines). In some embodiments, other influenza vaccines are peptide-based universal influenza vaccines (e.g., U.S. Pat. No. 7,354,589; and U.S. Pat. No. 7,527,798).

The disclosed immunogenic compositions and vaccines can be administered to individuals in need thereof, including humans, pigs, dogs, ferrets, and other mammals, to induce an immune response against strains of influenza A virus other than the strain(s) from which the components were derived. In some embodiments, administration follows a “prime-boost” regimen, in which at least a second dose (“boost”) of a vaccine is provided some time after the first (e.g., 1, 2, 3, 4, 5, 6, 7, 8, 9, or 10 weeks or long or 1, 2, 3, 4, 5, 6, 7, 8, 9, 10, 11, or 12 months or long after the first dose). The boost can be at the same dose or at a different dose. In any of these embodiments, either the same immunogenic composition or a different immunogenic composition can be administered. For example, a fusion protein or a modified hexon protein can be used for both the prime and the boost. In other embodiments, a fusion protein is used to prime, and a modified hexon protein is used for the boost. In other embodiments, a modified hexon protein is used to prime, and a fusion protein is used for the boost. In some embodiments, the prime is carried out with both the modified hexon protein and the fusion protein, and the boost is carried out with either the modified hexon protein or the fusion protein, or both.

In some embodiments, the Ad vector comprising the modified hexon protein and/or the fusion protein is administered. Typical dosage amounts of virus administered range from 10⁷-10¹¹ virus particles (e.g., 10⁷, 5×10⁷, 10⁸, 5×10⁸, 10⁹, 5×10⁹, 10¹⁰, 5×10¹⁰, 10¹¹). In some embodiments, the fusion protein or a nucleic acid molecule encoding the fusion protein is administered.

Methods of administration include, but are not limited to, mucosal (e.g., intranasal), intraperitoneal, intramuscular, intravenous, and oral administration. Immune responses can be assessed using suitable methods known in the art, including those taught in the specific examples, below.

Those skilled in the art will appreciate that there are numerous variations and permutations of the above described embodiments, that fall within the scope of the appended claims.

### Example 1

### Construction and In Vitro Screening of Vectors

1.1. Construction and Quality Control of Vectors

AdC6 and AdC68 vectors that carry the M2e sequence within the viral hexon can be prepared using standard techniques. Modifications of the Ad hexon are carried out using standard cloning methods and sequence verification, followed by insertion of an expression cassette into E1 and verification by Southern Blotting or sequencing. Placing of the M2e epitope into the hexon of AdC68 was guided by a crystal structure available for this molecule. AdC6 belongs to the same serotype and, but for variable regions, has a high degree of sequence homology to AdC68, which permits identification of R1 (referred to in Ser. No. 61/488,904 as “VR1”) as shown below in the comparison of the hexon sequences of R1 (referred to in Ser. No. 61/488,904 as “VR1”) and its flanking regions for AdC6 and AdC68. R1 (referred to in Ser. No. 61/488,904 as “Vr1”) is bolded, and the insertion site within AdC68 hexon is underlined.

AdC68
(SEQ ID NO: 1)
YNSLAPKGAPNTCQWTYKADGETATEKTYTYGNAPVQGINITKDGIQLGTD
AdC6
(SEQ ID NO: 2)
YNSLAPKGAPNSSQWEQAKTGNGGTMETHTYGVAPMGGENITKDGLQIGTD

The molecular viral clones can be rescued on HEK 293 cells. Once viral plaques become visible (in general after 5-14 days), cells are harvested and virus is released by freeze-thawing. The virus can then be expanded over several passages on HEK 293 cells. Once a large-scale stock has been produced (40-50 T75 flasks), virus can be purified, titrated, and quality controlled.

1.1.2. Purification

Ad vectors can be purified by two rounds of buoyant density ultracentrifugation on CsCl gradients followed by column purification (Bio-Gel P-6DG), then diluted in PBS supplemented with 10% glycerol and stored at −80° C.

1.1.3. Titration

Content of virus particles (vps) can be determined by spectrophotometry at 260 nm and 280 nm, with the latter determining purity of the preparation. Viral titer (vp/ml) can be determined using the formula: OD₂₆₀×dilution×1.1×10¹². The FDA requires dosing of adenoviral vectors according to vps as they determine the toxicity of the vectors.

Immunogenicity of the vectors on the other hand depends on the number of virus particles that are able to infect cells and to transcribe the transgene product. In general, numbers of infectious virus particles are measured by plaque assays using an agarose overlay or by an end-point dilution assay, which determines cytopathic effects. Both assays are performed on a cell line that provides E1 in trans. Plaque formation is dependent on a number of viral factors and may not reliably reflect the interactions between the cell line and the E1-deleted AdC vectors.

Two alternative assays can be used to determine the infectivity of adenoviral batches. The validity of the assays was checked against the standard plaque assay, and both assays show equal sensitivity. In one form of this assay the content of infectious virus particles is determined by nested RT-PCR with transgene or Ad (hexon) specific primers on RNA isolated from HEK 293 cells infected for 5-7 days with serial dilutions of vector. A standard is included to control the assay. This assay works for all of the AdC vectors.

In the second assay, HEK 293 cells are infected for 7 days with varied concentrations of the vector, then stained with an antibody to a conserved region of hexon and counterstained with a secondary alkaline phosphatase-labeled antibody. This assay is useful to detect Ad vectors with an unmodified hexon. It is not suited to detect Ad vectors with a R1 (referred to in Ser. No. 61/488,904 as “VR1”) modified hexon.

In other embodiments, staining with a monoclonal antibody to M2e is used to detect the hexon of M2e-modified vectors. HEK 293 cells are infected with varied concentrations of M2e hexon-modified vectors. Assay will be carried as described above but with a monoclonal antibody to M2e instead of the antibody against hexon.

1.1.4. Routine Quality Controls (QC)

Vectors may undergo a series of quality controls before they are released for animal testing. Vector batches can be checked for replication competent Ad (RCA) on A549 cells. Replication competent adenoviruses (RCA) can emerge during the creation and propagation of E1-deleted replication-defective Ad vectors in HEK 293 cells as a result of recombination between overlapping viral sequences in HEK 293 cells and vectors.

Depending on the level of RCA in vector preparations, it could have significant impact on vector performance, host immune responses, and toxicological profiles in in vivo experiments. Therefore, identification of vector preps with a high level of RCA contamination is important for gene transfer and virus-based vaccine applications of Ad vectors.

Briefly, cells in 6 well plates are treated with 2×10⁹, 2×10¹⁰, and 2×10¹¹ vps of vector. Control wells are infected with 10 or 50 plaque-forming units (pfu) of the corresponding wild-type Ad virus. In a third set of wells, the replication-defective vector (2×10¹¹ vp) is spiked with infectious virus (1, 10, and 100 pfu) to ensure that formation is not inhibited at the dose of vector plaque used. Cells are overlaid with agarose. Plates are read 4 and 8 days later. RCA commonly contaminate batches of E1-deleted human serotype 5 Ad (AdHu5) vectors grown on HEK 293 cells, but they have not yet been detected with E1-deleted AdC vectors transcomplemented with E1 of AdHu5 due to sequence differences in the E1 flanking regions.

Batches can be tested to detect and quantitatively determine the gram-negative bacterial endotoxin level in a test article. This can be carried out, for example, using the Limulus Amebocyte Lysate (LAL) gel-clot method and a commercial kit. Release criteria for vector lots to be used in large animal studies can be set, for example, at <5 endotoxin units (EU)/kg of animal weight, which is the parameter for humans set by the FDA.

Vector sterility can also be assessed. The purpose of this assay is to test for sterility of Ad vector preps by an inoculation/amplification and plating procedure. Briefly, control and testing articles are first inoculated and grown in LB medium overnight with agitation.

The cultures are then plated onto LB agar plates for a 48-hour incubation to detect formation of bacterial colonies and fungal growth. The control can be bacterial strain DH5α with serial dilutions and cultured under the same conditions.

The genetic integrity and identity of large-scale vector batches can be assessed by isolation of viral DNA. The recombinant DNA is digested with a set of restriction enzymes (in parallel to the molecular clone) and analyzed by gel electrophoresis. Because the disclosed vectors are created by constructing molecular clones and rescued/expanded in HEK 293 cells, original molecular clones and shuttle plasmids used for generating molecular clones can be used in side-by-side restriction digestions with viral DNAs extracted from vector preps to compare signature banding patterns by ethidium bromide stained agarose gel electrophoresis. In addition, molecular clones of vector backbones without transgene cassettes can be included in the analysis. At least two sets of restriction enzymes are usually selected for analysis. One set focuses on detecting presence and integrity of transgene cassettes; the other set emphasizes vector backbones. Genetic stability of vectors can be tested by Southern Blotting upon 12-15 serial passages on HEK 293 cells.

Expression of transgene product by Western Blot or immunoprecipitation can be tested upon infection of CHO cells stably transfected to express the Coxsackie adenovirus receptor (CHO-CAR) with 1,000 and 10,000 vps/cell of new vectors. Control cells are infected with the same dose of an Ad vector expressing an unrelated transgene product. Expression of modified hexon is measured by Western Blot of purified virus with a monoclonal antibody to M2e. An early passage master virus bank (40 vials of 0.5 ml each+10 vials of 0.1 ml) can be established, and vectors can be derived from this bank. Once this master virus bank has been depleted, infectious Ad vectors can be re-derived from the molecular clone to establish a new master virus bank. Genetic stability can be tested by serial propagation (15) of vectors followed by Southern Blotting to ensure that vectors do not undergo recombination.

1.1.5. Release Criteria

Yields of large-scale vector preparations are in general >10¹³ vps per batch (˜10⁸ HEK cells). Vp to infectious units ratios are commonly higher for AdC vectors than for human serotype Ad vectors and generally range between 1:20-1:200, but can range up >1:400.

### Example 2

### Immunogenicity and Efficacy of Vectors in Young Mice

Immunogenicity is assessed in groups of young (6-8 week old) C57Bl/6 mice (group size: 8 mice each). The prototype AdC68-3M2eNP and AdC6-3M2eNP vectors (referred to in Ser. No. 61/488,904 as “AdC68M2e(3)NP and AdC6M2e(3)NP vectors,” respectively) without hexon modifications have been tested extensively and are used for comparison.

2.1. Immunogenicity in Naïve Mice

The 4 vectors, i.e., AdC68-3M2eNP and AdC6-3M2eNP (referred to in Ser. No. 61/488,904 as “AdC68M2e(3)NP and AdC6M2e(3)NP vectors,” respectively) with and without the hexon modification are tested in a dose escalation experiment in which 8 young mice are injected with 10⁸, 10⁹, or 10¹⁰ vp of vector given intramuscularly. Vectors expressing an unrelated antigen, i.e., glycoprotein of rabies virus are used as negative controls. Mice are bled 2, 4 and 8 weeks after immunization.

Peripheral blood mononuclear cells (PBMC) are isolated and tested for frequencies of NP-specific CD8+ T cells by staining with an MHC class I tetramer specific for the immunodominant epitope of NP. Mice are euthanized three months after immunization.

Lymphocyte populations isolated from blood, spleen, and lungs are tested for frequencies of NP specific T cells by intracellular cytokine staining upon stimulation of cells with the NP peptide carrying the immunodominant MHC class I binding epitope in presence of brefeldin. Specifically cells are tested for production of IFN-γ, IL-2, TNF-α, MIP-1α, and perforin. Prior to intracellular staining, cells are surface stained for CD3, CD8, CD4, CD44, and CD62L. Stained cells are fixed with BD Stabilizing Fixative (BD Biosciences, San Jose, Calif.) and then analyzed by FACS using an LSR II benchtop flow cytometer (BD Biosciences, San Jose, Calif.) and FACSDIVA™ software. Flow cytometric acquisition and analysis of samples are performed on at least 100,000 events. Post-acquisition analyses are performed with FlowJo (TreeStar, Ashland, Oreg.). Single color controls with BD™ CompBeads Anti-Mouse IgiK (BD Biosciences, San Jose, Calif.) are used for compensation.

Plasma is tested for antibodies to M2e by ELISAs on plates coated with the M2e peptide and on plates coated with cells transfected with full-length M2 or sham-transfected. To further quantify B cell responses, cells isolated from spleen and bone marrow are tested for antibody-secreting cells (ASC). Ninety-six-well plates (IMMOBILON® P membrane; MAIPN4510; Millipore, Billerica, Mass.) are coated overnight at 4° C. with the M2e peptide at a concentration of 10 μg/ml in phosphate-buffered saline to detect M2e-specific ASC or with affinity-purified goat anti-human immunoglobulin A (IgA) plus IgG plus IgM (H+L; Kirkegaard & Perry, Gaithersburg, Md.) at a concentration of 4 μg/ml in PBS to determine overall frequencies of ASC. Plates coated with PBS are used as negative controls. Plates are incubated overnight at 4° C. and blocked for 2 h at 37° C. with RPMI 1640 medium supplemented with 10% fetal calf serum. PBMCs are suspended in RPMI medium supplemented with 10% FBS and 0.5 μg/ml of phosphatase-conjugated goat anti-human IgG (H+L) antibody. Cells are added at 2×10⁵ cells/well to the plates. They are incubated overnight at 37° C. The next day plates are washed and treated with alkaline phosphatase substrate. Numbers of ASC per well are determined by counting spots in an automated ELISpot reader. Data are recorded as spots per 10⁶ cells. M2e-specific ASCs are recorded as percentages of cells secreting antibodies to either of the strains of influenza A virus over cells secreting Ig.

ELISpot assays to test for memory B-cells are performed as described by Crotty et al. (17). PBMC are plated at 5×10⁵ cells/well in medium supplemented with pokeweed mitogen extract, 6 μg/ml of CpG oligonucleotide ODN-2006 (Invivogen, San Diego, Calif.), and 1/10,000 dilution of fixed Staphylococcus aureus Cowan (Sigma). Control wells do not receive mitogens. Cells are cultured for 5 days and then tested by an ELISpot assay for IgG secreting B cells and M2e-specific IgG-secreting B cells as described above. Spots from wells with control cells are subtracted from spots with mitogen stimulated wells. Otherwise data are recorded and quality controlled as described above. At euthanasia plasma are collected and tested again for antibodies to M2e by a peptide ELISA. Isotypes of antibodies are determined. The quality of antibodies are analyzed by BIACORE® affinity measurements. Plasma is tested for neutralizing antibodies to wild-type Ad vectors and hexon-modified Ad vectors.

2.2 Immunogenicity of Prime Boost Regimens

Selecting the more immunogenic of the two sets of vectors (i.e., with or without hexon modification) prime boost regimens are conducted using both AdC68 for priming followed by an AdC6 boost and vice versa, AdC6 priming followed by an AdC68 boost. AdC vectors expressing the rabies glycoprotein are used as negative controls. Mice are primed with 10⁸-10¹⁰ vp of vectors given intramuscularly; they are boosted two months later with the heterologous vector. Immune responses are assessed as described in 2.1.

2.3 Immunogenicity in Influenza Virus-Experienced Mice

In most adults influenza vaccination elicits largely a recall response of B and T cells to the more conserved antigens, which were induced by previous infections or vaccinations. As secondary responses follow different rules than primary and can in general be elicited by lower doses of antigens, a prime boost is carried out as described above (using only one of the regimens, i.e., either AdC68 followed by AdC6 or vice versa) in mice that were immunized at 6 weeks of age with 10′ TCID50 of influenza virus A/X31, a mouse attenuated H3N2 strain which only establishes an infection in the upper airways and therefore does not cause disease. They are prime-immunized 2 months later with the AdC vaccine, followed by a boost with the heterologous AdC vector. Doses of vectors and interval between priming and boosting are selected based on results of 2.2. Immune responses are monitored as described in 2.1.

2.4 Vaccine Efficacy in Young Mice

Protection upon a single dose of vaccine. These tests are conducted in two strains of mice: C57Bl/6 mice, which allow for testing of T and B cell responses; and ICR mice, which being outbred do not allow for testing of CD8+ T cell responses but provide a more realistic model for humans. Mice are immunized intramuscularly. The single immunization regimen is tested in naive and A/X31 pre-infected mice, and the duration of protection is determined. A heterologous H1N1 virus, influenza A Mammoth Fort Worth, (A/FM), which has already been titrated in mice to determine mean lethal dose, is used for challenge. The amount of challenge virus is varied. The viruses are used at 10 LD₅₀ in initial experiments. If case protection is achieved against this dose, the challenge dose is increased up to 1000 LD₅₀ to determine robustness of protection. Protection is assessed by measuring weight loss and morality (young animals are euthanized once they lose 30% of their original weight) as well as oxygen saturation on days 3, 5, and 7 following challenge. Lung virus titers are measured on days 4 and 7 following challenge. At the same times histology of one lung lobe are assessed to determine the degree of pathology.

Protection upon prime-boosting. Mice are immunized intramuscularly with varied concentration of the selected AdC vectors given in a 2 months interval intramuscularly. In subsequent tests this interval is changed to 4 and 6 months. One group is challenged 2 months after vaccination with 10 LD₅₀ of influenza A/FM; the other is challenged 8 months after vaccination. After vaccination titers of antibodies to M2e and frequencies of NP-specific CD8+ T cells are measured. Weight loss and mortality are determined. Provided that protection is achieved at both time points (i.e., survival of at least 80% of vaccinated mice with death of at least 80% of control mice), the test is repeated for challenge 8 months after vaccination.

Mice are euthanized 4 and 7 days after challenge and lung virus titers are determine by titration of the supernatants of lung homogenates on MDCK cells followed by a hemagglutination assay as described in Rowe et al., J Clin Microbiol. 37:937-43, 1999.). One lung section is used for histochemistry. Lungs are perfused with PBS and gently inflated with 200 μL of a 10% formalin solution through a 30 g needle. One inflated lung lobe is submerged in 10% formalin for tissue fixation for 24 hours at 4° C. Formalin-fixed lung samples are paraffin-embedded and sectioned at 4 m for mounting onto microscope slides. Sections are stained with H&E, and two sections of each lung are examined. Histopathological changes are examined by an investigator who is unaware of the samples' origin. Lung pathology is scored as follows: 1—no pathology; 2—perivascular infiltrates, 3—perivascular and interstitial infiltrates affecting <20% of the lobe; 4—perivascular and interstitial infiltrates affecting 20-50% of the lobe; 5—perivascular and interstitial infiltrates affecting >50% of the lobe. A regimen that induces protection in C57Bl/6 mice is tested (together with positive and negative control vectors) in young ICR mice to ensure that protection can be achieved in outbred mice.

Protection of a/X31 Pre-Exposed Mice

Most humans have immunological experience with influenza virus due to previous infections/vaccinations. To assess the effect of pre-exposure to A/X31 on the efficacy of the vaccine, ICR mice are infected intranasally with 1000 TCID50 of this virus; they are then immunized with the single dose regimen including control vectors and challenged with a high dose of A/FM virus. Protection is assessed by measuring weight loss, survival, and oxygen saturation.

Immunogenicity and efficacy in aged mice. Influenza is disproportionally fatal in the aged, which due to a general impairment of their immune system do not mount adequate responses. Unfortunately available influenza vaccines also show limited efficacy in the aged. Vaccine regimens are tested in 19-21 months old C57Bl/6 mice. To mimic pre-exposure to live influenza virus in humans, C57Bl/6 mice are infected at 8-9 months of age with a low dose (1000 TCID50) of A/X31 (H3N2) virus. In some embodiments, mice are primed at 19 months of age, boosted at 21 months of age, and challenged at 23 or 25 months of age. Ten mice per group are used for immunogenicity studies which are performed as described above, 20 mice per group are enrolled for challenge studies. Initial tests are conducted with low doses of A/FM challenge virus (3 LD₅₀). Challenge virus dose is gradually escalated (10, 100, 1000 LD₅₀) in some embodiments.

### Example 3

### Vaccine Efficacy in Larger Animals

Ferrets are highly susceptible to human strains of influenza virus and are viewed as a suitable pre-clinical model for influenza virus infection. An additional model is a nonhuman primate challenge model. The ferret study, which uses a contemporary H1N1 virus, is used at biosafety level 2; the virus for challenge of nonhuman primates, a highly pathogenic H5N1 virus, is conducted at biosafety level 3+ approved by CDC and USDA.

3.1. Ferret Model

Young ferrets (n=6) are vaccinated as described above. An additional 6 control animals are vaccinated with AdC vectors expressing the rabies virus glycoprotein. Vaccines are given at 10¹⁰ vp i.m. In case of a prime-boost regimen, animals are primed and the boosted. Timing between prime and boost is determined based on results from mouse studies. Sera are monitored in 2 week-intervals for antibodies to M2e. As a positive control, 6 additional ferrets are vaccinated with 1/10th of the human dose of the seasonal influenza vaccine (TIV) given at the time of priming of the other animals. Two months after the last AdC vaccine dose, ferrets are challenged with the 2009 H1N1 virus, which causes disease but not death in the species. After challenge ferrets are monitored for disease (fever, weight loss). Viral titers are measured from broncheal lavage on days 5 and 7 following challenge and serum are used to measure antibody responses to M2e and to the challenge virus.

3.2 Nonhuman Primate Model

Indian origin rhesus macaques (6 per group) are initially tested for neutralizing antibodies to the vaccine carrier. Only animals that lack such antibodies are enrolled. Animals are vaccinated with 10¹¹ vp of the AdC vaccines (expressing antigens of influenza virus or the rabies virus glycoprotein). They are boosted if indicated by mouse studies that increases vaccine efficacy with heterologous vectors. Timing between prime and boost is based on results from mouse studies. An additional 6 animals are enrolled and receive the contemporary influenza vaccine (TIV) at the human dose at the time of priming of the experimental animals. All animals are challenged with strain A/Vietnam/1203/2004 (H5N1), given at a concentration of 1×10⁶ 50% egg infectious dose intratracheally to sedated animals. Body weight, temperature and food intake are monitored twice daily, viral titers are measured from tracheal lavage on day 2, 4, 6 and 8. In case animals develop symptoms that necessitate their euthanasia, histopathology is assessed from HE-stained lung section.

Immunological responses are assessed after vaccination and on day 5 and 10 following challenge as follows. Blood is collected at days 0, 7, 21, 42 and 84 following each vaccine dose. Sera are tested for antibodies to M2e. Sera from positive control animals are monitored for antibodies to the corresponding stains by a microneutralization assay. PBMCs collected on days 0 and 7 are tested by ELISpot for M2e-specific antibody secreting cells. PBMCs collected at the other time points (as prior to vaccination) are tested for T cell responses to NP using an ICS as follows. PBMCs are stimulated with an NP peptide pool (15mer peptides overlapping by 10 amino acids are used at a final concentration of 2 μg of each peptide per ml). Cells are initially frozen so that assays for each individual animal can be conducted in parallel. Frozen cells are thawed and immediately washed with HBSS supplemented with 2 units/ml DNase I, resuspended with RPMI media and stimulated for 6 hrs with anti-CD28 (clone CD28.2), anti-CD49d (clone 9F10), and Brefeldin A. 14. Cells are stained with Violet-fluorescent reactive dye-Pacific Blue (Invitrogen, Carlsbad, Calif.), anti-CD14-Pacific Blue (clone M5E2), anti-CD16-Pacific Blue (clone 3G8), anti-CD8-APC-H7 (clone SKi), anti-CD4-Alexa700 (clone OKT4), anti-CD95-PE-Cy5 (clone DX2), and anti-CD28-Texas Red (clone CD28.2, Beckman Coulter, Fullerton, Calif.) for 30 min at 4° C. Additionally, cells are stained with anti-CCR7-PE (clone 150503) (frozen cells).

After fixation and permeabilization with CYTOFIX/CYTOPERM® (BD Biosciences, San Jose, Calif.) for 30 min at 4° C., cells are stained with anti-IFN-γ-APC (clone B27), anti-IL-2-FITC (clone MQ1-17H12), anti-TNF-α-PE-Cy7 (clone MAb11, R&D System) and anti-CD3-PerCp-Cy5.5 (clone SP34-2) for 30 min at 4° C. Cells are washed twice, fixed with BD Stabilizing Fixative (BD Biosciences, San Jose, Calif.), and then analyzed by FACS using an LSR II benchtop flow cytometer (BD Biosciences, San Jose, Calif.) and FACSDIVA™ software. Flow cytometric acquisition and analysis of samples are performed on at least 400,000 events. Post-acquisition analyses are conducted with FlowJo (TreeStar, Ashland, Oreg.). Post challenge sera are tested on day 10 following virus infection for neutralizing antibodies to the challenge virus.

### Example 4

### Materials and Methods for Examples 5-9

1. Construction of Hexon-Modified AdC68 Vectors

AdC68 vectors expressing the M2e peptide within hexon were generated as follows: a fragment encoding most parts of the hexon sequence and flanked with Mlu I was released from the E1-deleted viral molecular clone of AdC68 and cloned into the pcDNA3.1 vector (Invitrogen, Carlsbad, Calif.). The part of the M2e sequence of A/PR8/34 virus encoding LTEVETPIRNEWG (SEQ ID NO:3) was cloned into R1 of hexon after deletion of hexon residues 142-144 (ETA). To generate the R4 modified vector, the same M2e sequence was inserted between hexon residues 253 and 254.

Upon verification of the correct insertion of the M2e encoding base pairs by sequencing, the hexon sequence was excised from the pcDNA3.1 vector and cloned back into the viral molecular clone. For some vectors an expression cassette containing the previously described 3M2eNP sequence (42) under the control of the CMV early promoter was placed into E1. Recombinant viral molecular clones were used to rescue virus in HEK 293 cells. Virus was expanded on HEK 293 cells, purified by cesium chloride density-gradient centrifugation and virus particle (vp) content was determined by spectrophotometry at 260 nm. Vectors were titrated by a PCR based method to determine infectious units (IU).

Table 1 shows a list of the new vectors and the nomenclature used throughout this specification as well as pertinent growth characteristics such as yields per 10⁸ HEK 293 cells and vp to IU ratios. Other Ad vectors such as the AdC68-rab.gp vector, Ad vectors expressing GFP or AdC vectors expressing the 3M2eNP fusion protein have been described previously (37, 38) or were generated using previously described cloning techniques (43).

TABLE 1

| Name | Hexon Modification | Transgene | Yield | VP to IU Ratio |
|---|---|---|---|---|
| Ad68-HxM2eS(R1) | M2eS placed in R1 | None | 5.01 × 10¹³ | 111 |
| Ad68-HxM2eS(R4) | M2eS placed in R4 | None | 2.02 × 10¹³ | not tested |
| Ad68-3M2eNP-HxM2eS(R1) | M2eS placed in R1 | 3M2eNP fusion gene | 2.99 × 10¹³ | 108 |
| Ad68-3M2eNP-HxM2eS(R4) | M2eS placed in R4 | 3M2eNP fusion gene | 2.43 × 10¹³ | 50 |
| AdC68-3M2eNP | None | 3M2eNP fusion gene | 7.2 × 10¹³ | 150 |

2. Structure Modeling of AdC68 Modified Hexon

Models of AdC68, AdC68-HxM2eS(R1) and AdC68-HxM2eS(R4) hexon were generated using the Swiss-Model server (http site, swissmodel.expasy.org/). PyMOL V1.3 (Schro\ddot{d}inger LLC, Portland, Oreg., http site, pymol.org) was used to generate the customized 3D visualizations of the AdC68 hexon structures.

3. Expression of M2e on Viral Hexon

HeLa cells were infected with Ad vectors at 10²-10³ vps/cell. At 24 hours after infection, cells were harvested and stained with a monoclonal antibody to M2e (14C2-S1-4.2). After washing with PBS, the cells were incubated with a PE-labeled goat anti-mouse secondary antibody (Sigma, Ronkonkoma, N.Y.). Expression of M2e on the cells was then measured by flow cytometry.

As an alternative method to measure expression of M2e on hexon, Nunc 96-well plates were coated with 10¹⁰ vp of Ad vectors per well in 100 μl of coating buffer (15 mM Na₂CO₃, 35 mM NaHCO₃, and 3 mM NaN₃, pH 9.6) at 4° C. overnight. Plates were blocked with PBS containing 5% BSA at room temperature for 1 hour. Plates were then treated for 1 hour with serially diluted M2e monoclonal antibody (14C2-S1-4.2) at room temperature followed by incubation with the alkaline phosphatase-conjugated goat anti-mouse immunoglobulin and then the substrate.

4. Identification of the Encoded Transgene Product

To identify presence of the 3M2eNP fusion protein, cell lysates from virus-infected HEK 293 were prepared and proteins were separated by gel electrophoresis and transferred to a membrane, which was then blotted with a monoclonal antibody to M2e (14C2-S1-4.2).

5. Influenza Virus

Influenza virus A/PR/8/34 was grown in the chorioallantoic fluid of embryonated chicken eggs and titrated in adult mice upon their intranasal infection to determine the mean lethal dose (LD₅₀).

6. Mice

Female C57Bl/6 and ICR mice were purchased at age 6-8 weeks from ACE Animals (Boyertown, Pa.). All animal procedures reported herein were based on approved institutional protocols.

7. Immunization of Mice

Groups of 5-10 mice were vaccinated intramuscularly with a total of 1×10¹⁰ vp of the Ad vectors. Two months later, some groups of mice were boosted intramuscularly with the same or a heterologous vector given intramuscularly at the same dose.

8. Antibody Responses to M2e

Antibody responses specific to M2e were measured from sera of individual mice by an M2e-peptide ELISA using a previously published procedure (23). Briefly, the multiple antigenic peptide consisting of a Cys-(Gly-Lys)₃-Ala backbone (CGKGKGKA; SEQ ID NO:4) with two attached M2e(2-24) peptides was used to coat wells of Nunc 96-well plates (Thermo Fisher Scientific, Rochester, N.Y.) by incubating 50 μl of the peptide dilution at 85 nM in 0.02 M NaCl at 4° C. overnight. Plates were blocked for 2-18 hours with PBS containing 5% BSA. After washing, the plates were incubated for 1 hour with serial dilutions of sera in PBS+5% BSA followed by a one hour incubation with a 1:200 dilution of alkaline phosphatase-conjugated goat anti-mouse immunoglobulin (Cappel, Irvine, Calif., USA) for 1 h at room temperature. After being washed, plates were incubated for 20 min with substrate (10 mg d-nitrophenyl phosphate disodium dissolved in 10 ml of 1 mM MgCl₂, 3 mM NaN₃, and 0.9 M diethanolamine, pH 9.8) and then read in an automated ELISA reader at 405 nm. The assay was standardized with the monoclonal antibody to M2e (14C2-S1-4.2).

Antibody titers were determined by a previously described cellular ELISA (42). Briefly, 293T cells were infected with a lentivirus expressing the full-length M2 sequence of A/PR8/34 virus to generate stable M2⁺ cell lines. A control cell line was generated by infection of 293T cells with empty lentivirus. These cell lines were used as immunosorbents in an enzyme-linked immunosorbent assay as described (42). The assay was standardized with the 14C2-S1-4.2 antibody.

9. Antibody Responses to Ad Vectors

Ad-specific neutralization titers were measured on HEK 293 cells infected with AdC68 vectors expressing EGFP (AdC68-EGFP), as described previously (37). Briefly, a dose of AdC68EGFP (with or without hexon modifications) that caused EGFP expression in 70 to 90% of the cells within 24 h was chosen. Sera from mice vaccinated with 1×10¹⁰ vps of vectors were harvested 5 weeks after vaccination, and inactivated at 55 C for 30 min. Serial diluted sera were then mixed with appropriate doses of AdC68EGFP and incubated for 60 min at room temperature. The vector-serum mixture was mixed with an equal volume of HEK 293 cells at 10⁶ cells/ml and the mixture was transferred into flat-bottom 96-well plates. The plates were incubated overnight at 37° C. and then screened visually for green fluorescent cells under a UV microscope. The titer was determined as the reciprocal serum dilution that caused 50% reduction of fluorescent cells in comparison to that seen in control wells infected with vector only.

10. Tetramer Staining of T Cell

MHC class I NP peptide tetramer (ASNENTE™; SEQ ID NO:5) conjugated with APC was provided by the Tetramer Core Facility (Emory University, Atlanta, Ga.). Lymphocytes were stained with the NP tetramer, a PerCP-Cy5.5-labeled antibody to CD8 and a live cell stains (both from BD Biosciences, San Jose, Calif.) for 30 minutes at 4° C. Flow cytometric acquisition and analysis of samples was performed on at least 500,000 events. The post-acquisition data were processed using FlowJo 7.1.1 (TreeStar, Ashland, Oreg.).

11. Influenza Virus Challenge

Two months after vaccination, mice were anesthetized and then challenged intranasally with 10 LD₅₀ of influenza A/PR/8/34 virus diluted in 30 μl phosphate-buffered saline. Mice were monitored daily for weight loss and survival after challenge. Mice were euthanized once they lost in excess of 30% of their pre-challenge weight.

12. Statistical Analyses

Samples were tested by ELISA and neutralization assay in duplicate from individual mice. Tetramer staining was conducted with lymphocytes from individual mice. The comparison of means in different groups was determined by analysis of variance. The statistical significance of protection of vaccinated groups compared to the control group was determined using Fisher's exact test. Data with P≦0.05 are viewed as showing a statistically significant difference.

### Example 5

### Construction of Hexon-Modified AdC68 Vectors

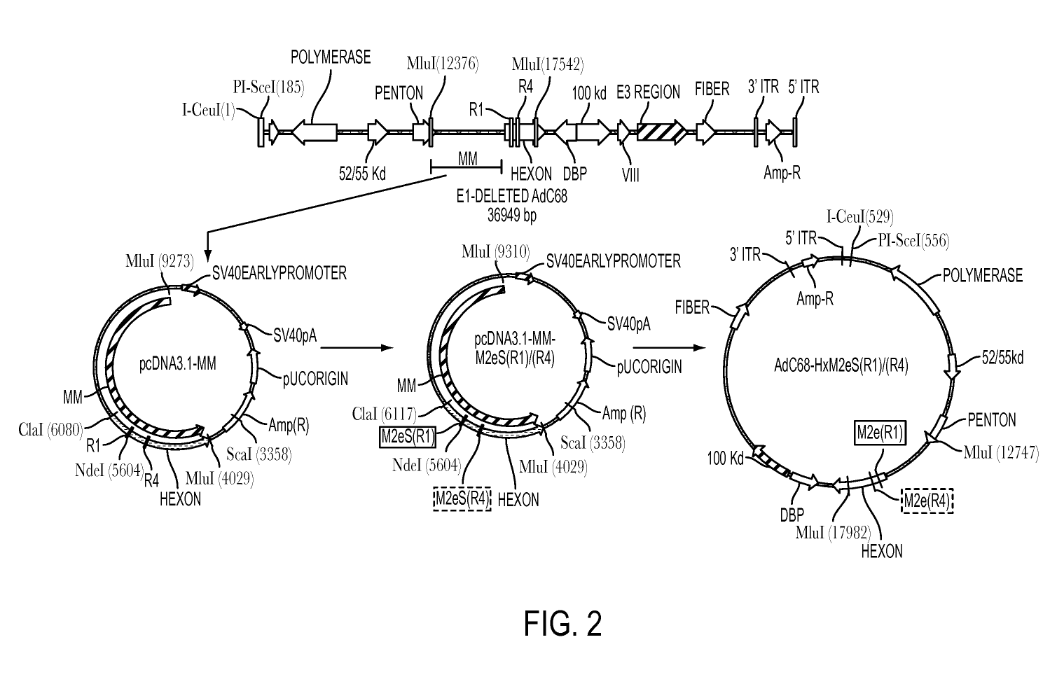

The hexon was modified by direct cloning of the M2e sequence into a segment of the viral molecular clone as shown in FIG. 2. Briefly the AdC68 molecular clone was digested with Mlu I, releasing a 5.1 kb fragment that contains most of the hexon sequence. The fragment was ligated into the Mlu I site of pcDNA3.1, resulting in plasmid pcDNA3.1-MM. 5′ oligonucleotides containing the Cla I site of hexon followed by the adjacent hexon sequences and the M2e sequence in position 142-144 of hexon; and 3′ primers containing the Nde I site of hexon were used to amplify a fragment that was then cut with Cla I and Nde I and cloned into the corresponding sites of pcDNA3.1-MM resulting in a plasmid containing M2e within R1 of hexon. To construct the R4 modified hexon, 5′ oligonucleotides containing the Nde I site of hexon followed by M2e in position 253 to 254 of hexon and 3′ oligonucleotides containing the Sca I site and adjacent sequences of pcDNA3 were used to amplify a fragment of pcDNA3.1-MM. The amplicon was cut with Nde I and Sca I and cloned into the corresponding sites of pcDNA3.1-MM resulting in a plasmid the contained M2e within hexon R4. Parts of the vectors were sequenced to ensure insertion of the M2e sequence. The hexon sequences were then cloned back into the viral molecular clone using Mlu I. The genomes of new vectors were analyzed by Southern Blotting to ensure correct insertion of the sequence.

### Example 6

### Structure Modeling of M2e-Modified AdC68 Hexon

AdC68 hexon in its native structure forms trimers with the variable loops encoded by R1-R5 displayed on the top of the molecule. To assess the effect of the M2e insertion into R1 or R4 of hexon, we modeled the structure of wild-type AdC68 hexon, which has been characterized in depth by X-ray crystallography (39) in comparison to M2e-modified hexon. Structural modeling predicted that native hexon and hexon with the R1 M2e insert would form trimers, whereas insertion of M2e into R4 was predicted to disrupt the structure and prevent hexon trimerization.

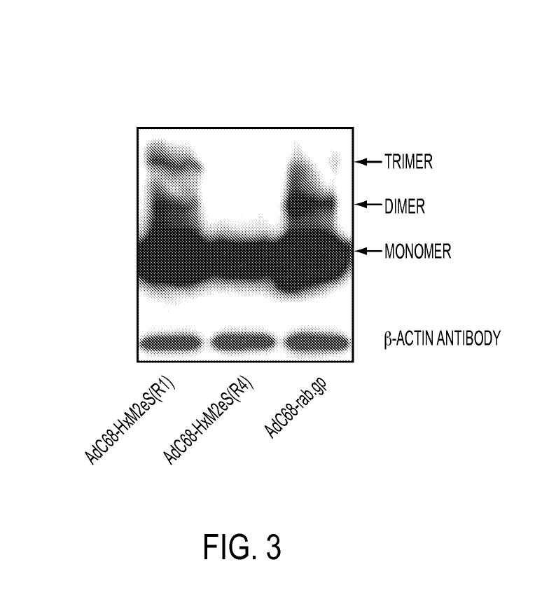

To further assess the reliability of the structure prediction, we isolated hexon under non-reducing conditions from AdC68 (with and without hexon modifications)-infected cells and conducted Western Blots with an Ad hexon-specific antibody. As shown in FIG. 3, most of the native hexon such as present on the previously described AdC68ab.gp vector (which carries native hexon) or the R1 modified hexon formed trimers, and only small fractions were isolated as monomers or dimers. Hexon molecules with an R4 modification on the other hand were exclusively isolated as monomers, which surprisingly interestingly did not prevent the vector from expanding in HEK 293 cells (Table 1).

### Example 7

### Expression of M2e on Hexon

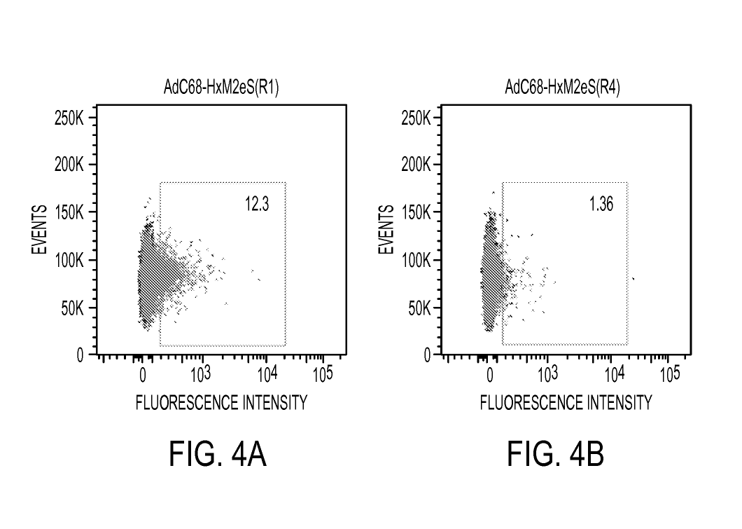

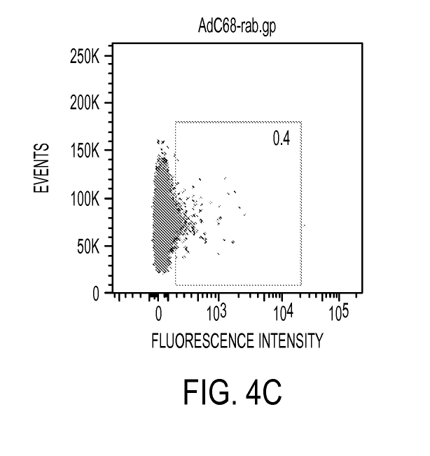

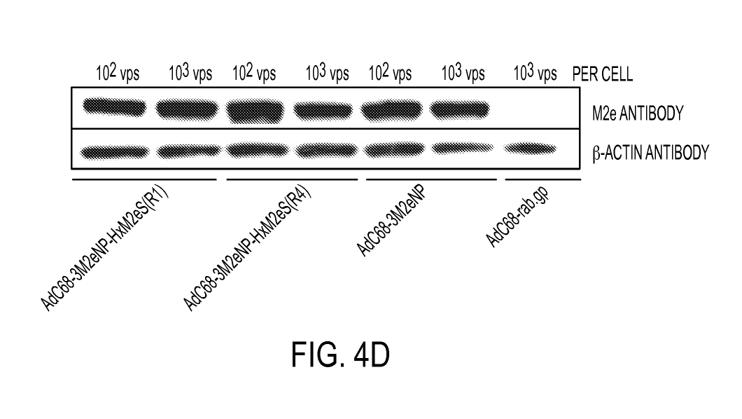

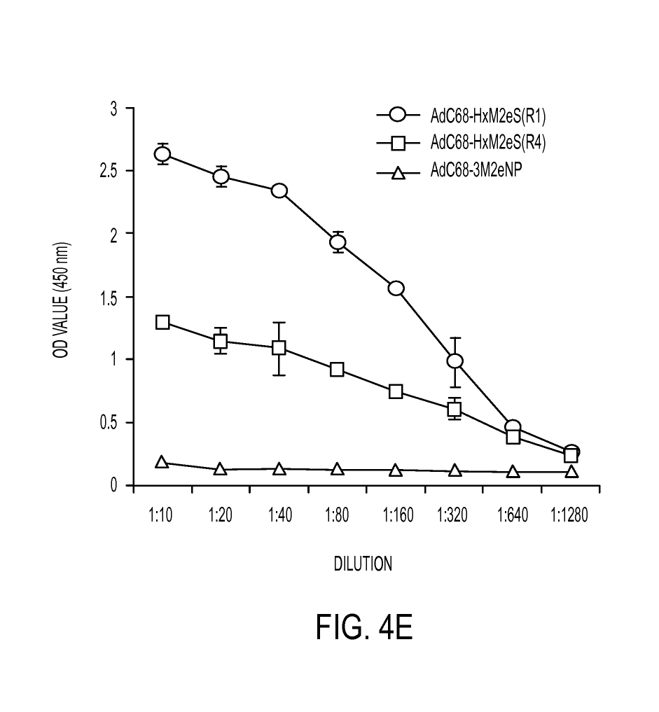

We used three methods to measure expression of M2e as expressed by hexon or encoded by the transgene, which also contains NP. In the first method, cells were infected with different amounts of Ad vectors and then stained with an antibody to M2e and a second PE-labeled antibody to mouse immunoglobulin. Cells were analyzed by flow cytometry. This method detects only cell surface expressed M2e and thus favors detection of M2e carried by hexon rather than M2e present on the transgene product, which due to a signal sequence is secreted from the cells. As shown in FIG. 4A, M2e could be detected on the surface of cells transduced with AdC68-HxM2eS(R1), levels were markedly lower on cells infected with AdC68-HxM2eS(R4) but still above those on cells infected with a control vector. Cells transduced with the AdC68-3M2eNP vectors with native hexon did not stain with the antibody.

To further quantify expression of M2e on virions we conducted ELISAs on plates coated with hexon-modified or native hexon AdC68 vectors, which were probed with a monoclonal antibody to M2e. As shown in FIG. 4B the M2e antibody showed high reactivity to the capsid of R1-modified vector and comparatively lower activity against the R4-modified capsid thus confirming results obtained with flow cytometry. The higher binding of the M2e antibody to the R1-modified hexon suggests that the loop encoded by R1 is more accessible to antibodies, which is consistent with the finding that this region contains the dominant neutralizing B cell epitope of AdC68 (25).

To assess transgene product expression, cells were infected with the Ad vectors expressing the 3M2eNP fusion protein (with or without hexon modifications). The following day, cell lysates were tested with the M2e antibodies by Western Blot. As shown in FIG. 4C, vectors expressed equal amounts of the M2e antibody binding protein that had the predicted size of the transgene product.

### Example 8

### Hexon R1 Modifications Escape Neutralization by Antibodies to Native AdC68

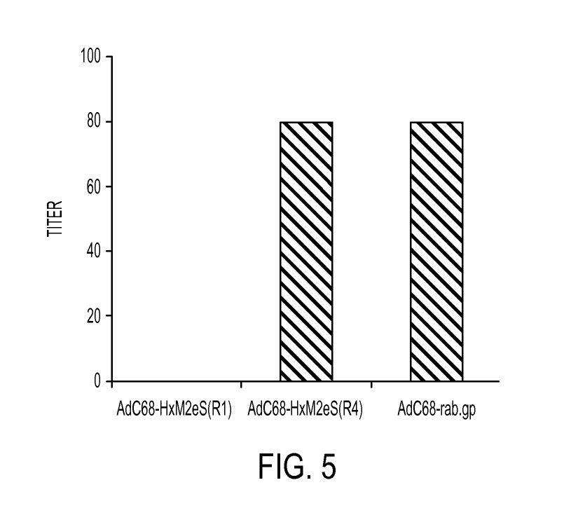

To test whether the R1 or R4 hexon modifications perturb binding of neutralizing antibodies to native hexon, mice were immunized with AdC68 vectors expressing native or M2e-modified hexon. Mouse sera were then tested for neutralization of an AdC68 vector expressing native hexon. As shown in FIG. 5A, sera from mice immunized with AdC68 vector expressing native hexon (AdC68-rab.gp) or R4 hexon-modified readily neutralized wild-type AdC68 virus while sera from mice immunized with the R1 hexon-modified vector neutralized the homologous vector but not the vector with native hexon.

In a second set of experiments we tested sera from mice immunized with an AdC68 vector expressing native hexon for neutralization of hexon-modified vectors. Sera showed equal titers when probed with AdC68 vector expressing native or R4-modified hexon but failed to neutralize AdC68 vectors with the R1 hexon modification. These results confirmed our previous studies (25), which had identified the sequence encoded by R1 as the major binding site of neutralizing antibodies to this virus.

### Example 9

### M2e-Specific Antibody Responses

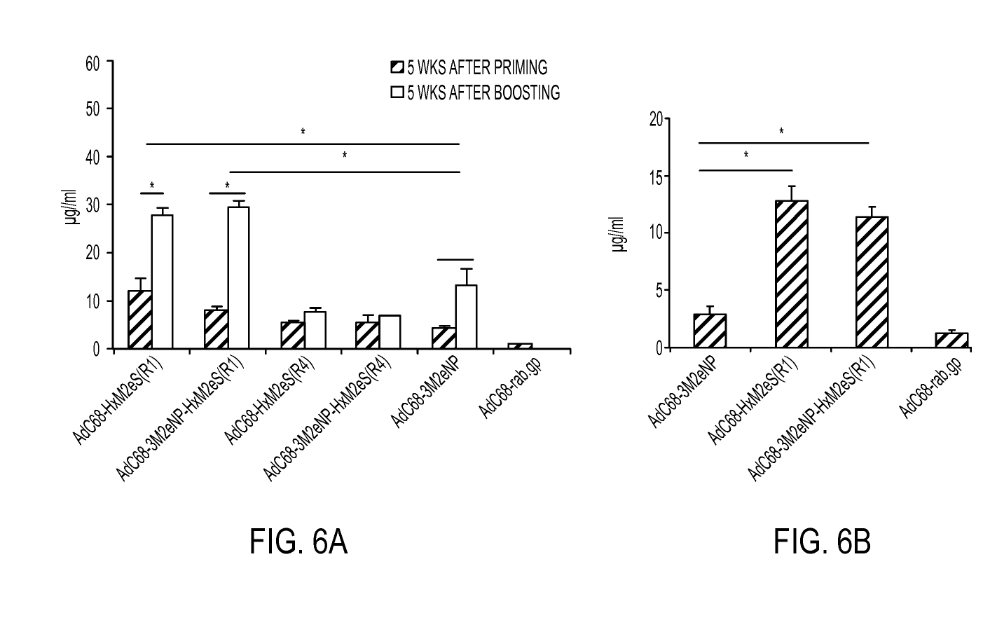

Groups of ICR mice were vaccinated with 1×10¹⁰ vp of recombinant AdC68 vectors and boosted 2 months later with same vector used at the same dose. For comparison, mice were vaccinated with the same dose of AdC68-3M2eNP; these mice were boosted with the heterologous AdC6 vectors expressing the same transgene product. A heterologous vector was used to prevent blunting of the recall response by vector-specific neutralizing antibodies induced upon priming. Sera were harvested from individual mice 5 weeks after the prime and the boost, respectively. Sera from mice immunized with vectors expressing the rabies virus glycoprotein served as controls. Sera were tested for antibodies to M2e by a peptide ELISA. See FIG. 6A.

All vectors expressing M2e either within hexon or from a transgene product induced antibodies to M2e. Responses were higher upon immunization with the R1 hexon-modified vector compared to vectors expressing M2e within R4 or as a transgene. Antibody titers increased markedly after the boost in mice immunized with the R1 hexon-modified vector, while increases in mice immunized with the R4 hexon-modified vector were modest. Immunization with the AdC68-HxM2eS(R1) vector given twice resulted in higher antibody responses to the M2e peptide compared to the heterologous AdC68-3M2eNP/AdC6-3M2eNP vaccine regimen. The presence of M2e within the transgene product did not, as we had expected, increase antibody responses to M2e. To ensure that the vaccine induced a response in a genetically distinct strain of mice, inbred C57Bl/6 mice were tested using the same vaccine regimens; the results were similar.

Antibodies to M2e peptides may not necessarily bind native M2e as expressed by influenza virus or on influenza virus-infected cells (23). We therefore also tested sera from C57Bl/6 mice immunized with 10¹⁰ vp of AdC68-3M2eNP, AdC68-HxM2eS(R1) or AdC68-3M2eNP-HxM2eS(R1) for antibodies in a cellular ELISA, which more faithfully detects antibodies to M2e as expressed within hexon. See FIG. 6B. The AdC68-3M2eNP vector only induced a marginal antibody response of <2 μg of M2e-specific antibodies per ml of serum. In contrast the R1 hexon-modified vectors induced titers of ˜10 μg/ml. Again, concomitant expression of the 3M2eNP fusion protein by the M2e R1 hexon-modified vector failed to increase antibody responses.

### Example 10

### NP-Specific CD8⁺ T-Cell Responses

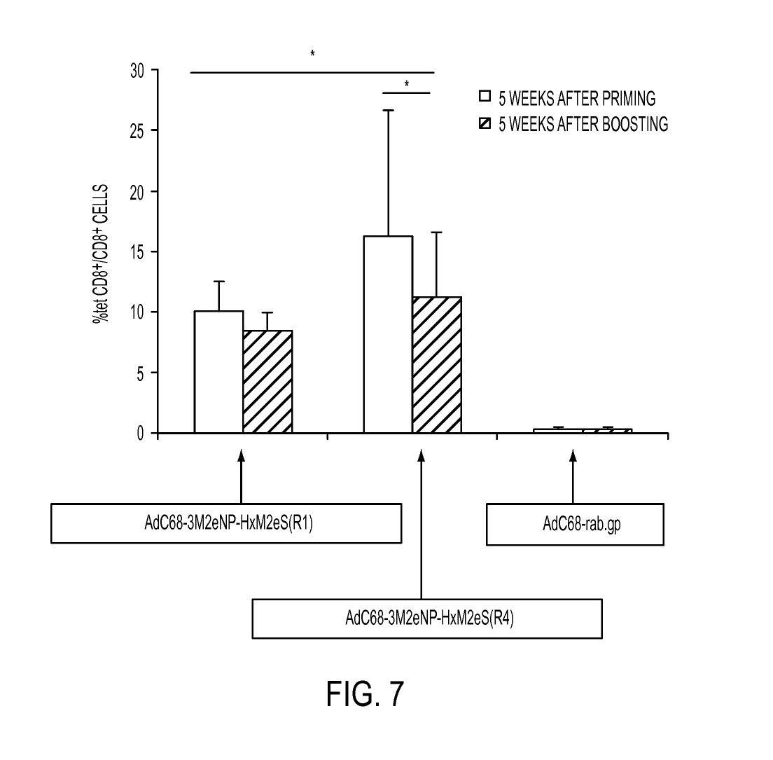

CD8⁺ T-cell responses to NP were tested at different time points after vaccination of C57Bl/6 mice with the AdC68-3M2eNP-HxM2eS(R1) or AdC68-3M2eNP-HxM2eS(R4) vectors from blood. After priming, all of the mice developed detectable frequencies of NP-specific CD8⁺ T cells, which were comparable to those previously reported for mice immunized with the hexon unmodified AdC68-3M2eNP vector (42). A booster immunization with the same vectors given 2 months after priming failed to increase circulating NP-specific CD8⁺ T cell frequencies, indicating that antibodies to the vaccine carrier impaired uptake of the vectors and thus expression of the transgene product. See FIG. 7.

### Example 11

### Protection Against A/PR8/34 Challenge

We conducted two sets of experiments to determine vaccine efficacy. In the first experiment, inbred C57Bl/6 mice were vaccinated with hexon-modified vectors with or without the 3M2eNP fusion protein. In the second experiment, the same vectors were tested in ICR mice together with the AdC68-3M2eNP vector carrying native hexon. In both experiments, mice vaccinated with the AdC68rab.gp vector were used as controls.

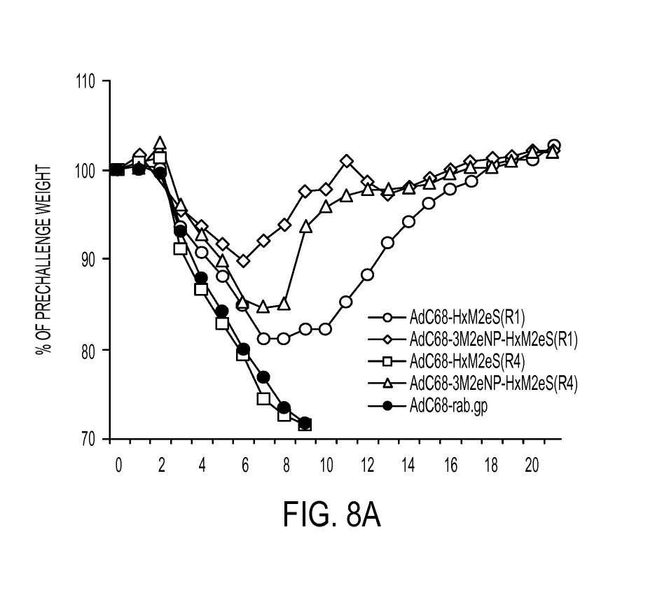

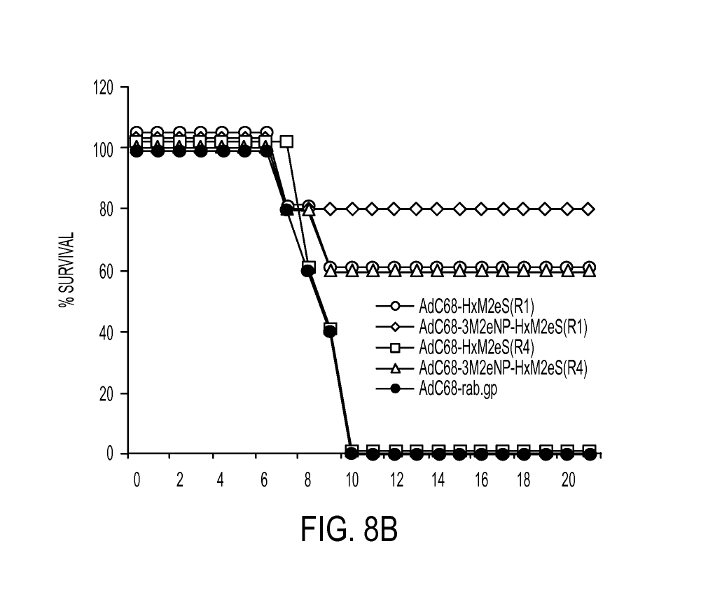

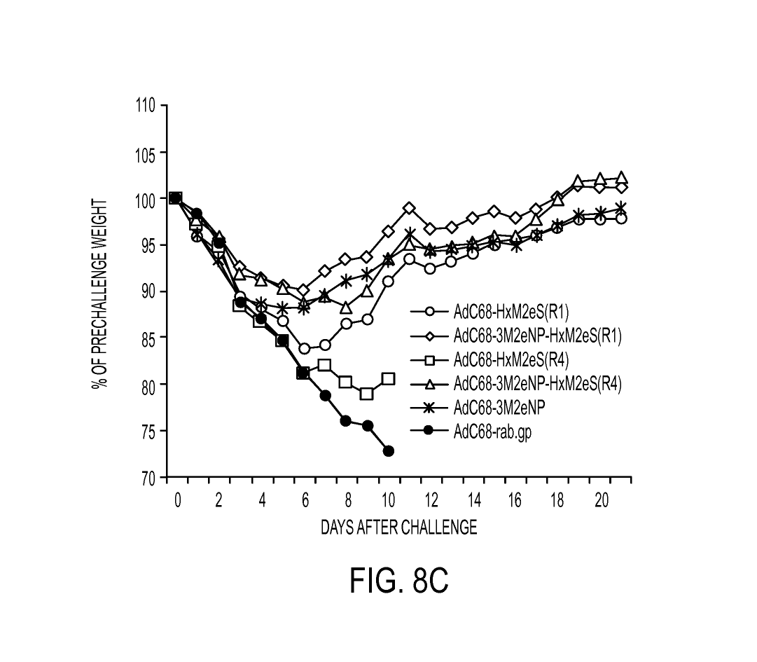

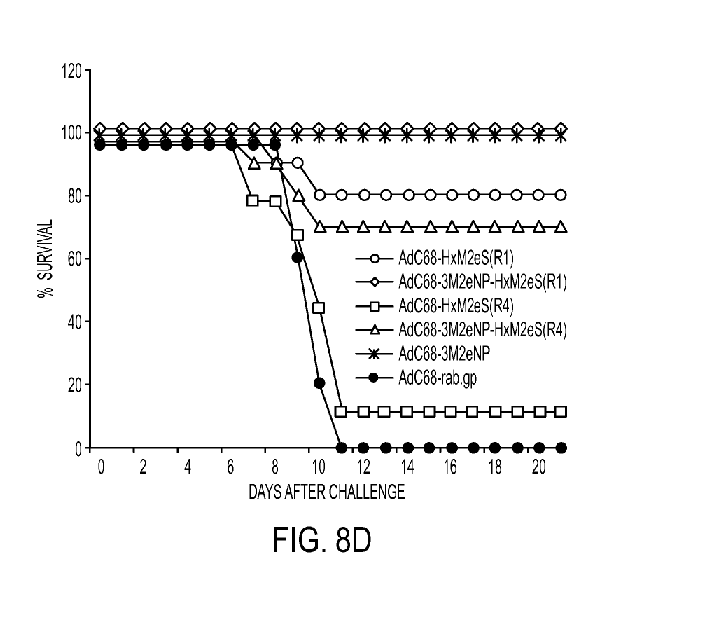

In the first experiment, vaccinated C57Bl/6 mice (5 per group) were infected 2 months after booster immunization with 10LD₅₀ of A/PR8/34 virus. Weight loss of vaccinated mice peaked by days 6-8 after challenge and then most mice began to gain weight. By 21 days after challenge most protected mice had returned to their pre-challenge weight. Sham-vaccinated control mice continued to lose weight after challenge until they died or required euthanasia (FIG. 8A). Upon challenge, 80% (p=0.0238) of the mice vaccinated with AdC68-3M2eNP-HxM2eS(R1) survived, while 60% of the mice vaccinated with either AdC68-HxM2eS(R1) or AdC68-3M2eNP-HxM2eS(R4) survived (p>0.05, FIG. 8B). All mice in the AdC68-HxM2eS(R4) vaccine and control groups died.

The experiment was repeated with ICR mice (n=10). For this experiment a group receiving a previously described regimen composed of AdC68-3M2eNP vector priming followed by a boost with the AdC6-3M2eNP vector was included for comparison (42). Mice immunized with the AdC68-3M2eNP-HxM2eS(R1) vector twice or the AdC68-3M2eNP/AdC6-3M2eNP combination showed minimal weight loss of ˜10%, and all of the mice survived (p=0.0001). The AdC68-HxM2eS(R1) and AdC68-3M2eNP-HxM2eS(R4) vaccines also provided significant protection to 80% (p=0.0004) and 70% (p=0.0015) of mice respectively. Only one of the AdC68-HxM2eS(R4) immunized mice survived, and all of the mice of the control group succumbed the infection. Weight loss in general corresponded to level of protection against death except that mice immunized with the AdC68-HxM2eS(R1) vector, which on average lost more weight than mice immunized with the AdC68-3M2eNP-HxM2e(R4) vector.

### DISCUSSION

Adenovirus hexon is the most abundant of the viral capsid proteins forming a total of 240 trimers on the surface of the icosahedral capsid. Hexon molecules contain a pseudo-hexagonal base that is anchored to the capsid, a conserved barrel domain followed by a tower on top of the molecule that contains flexible loops (28). Different serotypes of Ad viruses show sequence variations mainly within these loops (29). AdC68 hexon, which has been characterized by X-ray crystallography (39), contains 5 variable regions (R1-5) that form five distinctive loops on top of the molecule. The loop encoded by R1 was defined as the dominant target of AdC68 neutralizing antibodies (25).

Ad vectors derived from the common human serotype 5 (AdHu5) displaying B cell epitopes from other pathogens within their hexon have been described previously and shown immunogenicity in mice (21, 36). Neutralizing antibodies to AdHu5 virus are common in humans and dampen uptake of AdHu5 vectors and hence immune responses to vector encoded transgene products (13), although they would not necessarily be expected to affect B cell responses to an epitope displayed within the viral hexon. It has been suggested that modification of the variable regions of Ad hexon prevents neutralization by antibodies to wild-type virus (1) but such results remain debatable (6, 26). As heterotypic protection against influenza A virus by antibodies to M2e is increased by concomitant stimulation of CD8⁺ T cells to NP (42), we opted to base the vaccine on a chimpanzee Ad vector, i.e., AdC68, to which most humans lack neutralizing antibodies (38). We inserted the M2e epitope into either R1 or R4 of AdC68 hexon. Additional vectors were constructed that carried the M2e hexon modifications and expressed a fusion protein composed of NP and 3 different M2e sequences as a transgene product. Vectors were tested in comparison to vectors carrying wild-type hexon for immunogenicity and efficacy against influenza A virus infection in mice.

The working examples above demonstrate that vectors with wild-type or modified hexon induce comparable CD8⁺ T cell responses in mice. Antibody responses to M2e were markedly higher upon immunization with the hexon-modified vectors that carried M2e within R1.

Insertion of the epitope into R1 did not appear to alter the overall structure of hexon as the R1 modified hexon could still form trimers on the virus capsid. In contrast, insertion of the same sequence into R4 prevented trimer formation. M2e present within R1 induced a more potent M2e-specific antibody response than the same sequence within R4. Without being bound by this explanation, we think that the loop encoded by R1 is more accessible to antibodies compared to the loop encoded by R4, as the former also carries the binding sites for the majority of neutralizing antibodies directed to native hexon (25). Nevertheless, we cannot rule out alternative explanations such as a role of a trimeric structure in optimizing B cell responses or differences in the secondary structure of the M2e epitope placed into either loop.

The AdC68 vector carrying the M2e sequence within the loop encoded by R1 also induced higher antibody responses especially to its native confirmation within M2 as compared to a transgene product composed of a fusion protein of 3 M2e sequences and NP. It is likely that the amount of a transgene product that is produced for at least 7-10 days under the control of the potent CMV promoter until vector-transduced cells have been eliminated by the immune system (40) would be well in excess to that of an antigen present on the capsid that is not or only at small amounts synthesized in vivo by an E1-deleted Ad vector. The higher immunogenicity of M2e as displayed on the viral capsid may reflect that B cell responses to rigidly arranged epitopes are less dependent on T help as has been shown previously in the vesicular stromatitis virus system (2) and that T help is limited upon immunization with an AdC vector. Considering that AdC vectors carry a number of antigens with potential MHC class II epitopes (34), we favor the alternative explanation that a more structured display of antigen favors B cell stimulation compared to antigen primarily present in an unordered fashion. Surprisingly we were unable to further increase M2e-specific antibody responses by displaying M2e on hexon and within the same vectors encoding M2e as part of the transgene product. This was observed with both R1 and R4 hexon-modified vectors and is thus unlikely to reflect antigen saturation, because the R4 hexon-modified vector only induced low antibody responses to M2e. B cell responses induced by M2e within R1 could easily be boosted by a second immunization with the same vector, which may mean that M2e had replaced the main neutralizing B cell epitope of AdC68. In contrast the R4 hexon-modified vector only elicited a marginal antibody recall response presumably due to interference by neutralizing antibodies to native parts of hexon. AdC vector-induced antibodies to M2e in absence of cellular immune responses to influenza virus provided partial protection against A/PR8/34 challenge. As reported previously (42), protection was improved by concomitant activation of NP-specific CD8⁺ T cell responses through a transgene product. Booster immunization with the homologous capsid modified Ad vectors failed to increase frequencies of NP-specific CD8⁺ T cell responses.

### REFERENCES

1. Abe et al., 2009. Adenovirus type 5 with modified hexons induces robust transgene-specific immune responses in mice with pre-existing immunity against adenovirus type 5. J Gene Med 11:570-9.
2. Bachmann et al., 1995. T helper cell-independent neutralizing B cell response against vesicular stomatitis virus: role of antigen patterns in B cell induction?Eur J Immunol 25:3445-51.
3. Barnes et al., 2012. Novel adenovirus-based vaccines induce broad and sustained T cell responses to HCV in man. Sci Transl Med 4:115ra1.
4. Bayer et al., 2010. Vaccination with an adenoviral vector that encodes and displays a retroviral antigen induces improved neutralizing antibody and CD4+ T-cell responses and confers enhanced protection. J Virol 84:1967-76.
5. Bett et al., 2010. Comparison of T cell immune responses induced by vectored HIV vaccines in non-human primates and humans. Vaccine 28:7881-9.
6. Bradley et al., 2012. Adenovirus serotype 5 neutralizing antibodies target both hexon and fiber following vaccination and natural infection. J Virol 86:625-9.
7. Carragher et al., 2008. A novel role for non-neutralizing antibodies against nucleoprotein in facilitating resistance to influenza virus. J Immunol 181:4168-76.
8. De Filette et al., 2005. Universal influenza A vaccine: optimization of M2-based constructs. Virology 337:149-61.
9. Ekiert et al., 2009. Antibody recognition of a highly conserved influenza virus epitope. Science 324:246-51.
10. Ekiert et al., 2011. A highly conserved neutralizing epitope on group 2 influenza A viruses. Science 333:843-50.
11. Epstein, 2006. Prior H1N1 influenza infection and susceptibility of Cleveland Family Study participants during the H2N2 pandemic of 1957: an experiment of nature. J Infect Dis 193:49-53.
12. Fan et al., 2004. Preclinical study of influenza virus A M2 peptide conjugate vaccines in mice, ferrets, and rhesus monkeys. Vaccine 22:2993-3003.
13. Fitzgerald et al., 2003. A simian replication-defective adenoviral recombinant vaccine to HIV-1 gag. J Immunol 170:1416-22.
14. Gschoesser et al., 2002. CD4+ and CD8+ mediated cellular immune response to recombinant influenza nucleoprotein. Vaccine 20:3731-8.
15. Hensley et al., 2005. Dendritic cell maturation, but not CD8+ T cell induction, is dependent on type I IFN signaling during vaccination with adenovirus vectors. J Immunol 175:6032-41.
16. Huleatt et al., 2008. Potent immunogenicity and efficacy of a universal influenza vaccine candidate comprising a recombinant fusion protein linking influenza M2e to the TLR5 ligand flagellin. Vaccine 26:201-14.
17. Jimenez et al., 2007. Vaxfectin-formulated influenza DNA vaccines encoding NP and M2 viral proteins protect mice against lethal viral challenge. Hum Vaccin 3:157-64.
18. Krause et al., 2006. Epitopes expressed in different adenovirus capsid proteins induce different levels of epitope-specific immunity. J Virol 80:5523-30.
19. Lalor et al., 2008. Plasmid DNA-based vaccines protect mice and ferrets against lethal challenge with A/Vietnam/1203/04 (H5N1) influenza virus. J Infect Dis 197:1643-52.
20. Liu et al., 2004. Monoclonal antibodies recognizing EVETPIRN epitope of influenza A virus M2 protein could protect mice from lethal influenza A virus challenge.

Immunol Lett 93:131-6.
21. Matthews et al., 2010. HIV antigen incorporation within adenovirus hexon hypervariable 2 for a novel HIV vaccine approach. PLoS One 5:e11815.
22. McMichael et al., 1986. Recognition of influenza A virus nucleoprotein by human cytotoxic T lymphocytes. J Gen Virol 67 (Pt 4):719-26.
23. Mozdzanowska et al., 2003. Induction of influenza type A virus-specific resistance by immunization of mice with a synthetic multiple antigenic peptide vaccine that contains ectodomains of matrix protein 2. Vaccine 21:2616-26.
24. Neirynck et al., 1999. A universal influenza A vaccine based on the extracellular domain of the M2 protein. Nat Med 5:1157-63.
25. Pichla-Gollon et al., 2007. Structure-based identification of a major neutralizing site in an adenovirus hexon. J Virol 81:1680-9.
26. Pichla-Gollon et al., 2009. Effect of preexisting immunity on an adenovirus vaccine vector: in vitro neutralization assays fail to predict inhibition by antiviral antibody in vivo. J Virol 83:5567-73.
27. Reyes-Sandoval et al., 2004. Human immunodeficiency virus type 1-specific immune responses in primates upon sequential immunization with adenoviral vaccine carriers of human and simian serotypes. J Virol 78:7392-9.
28. Roberts et al., 1986. Three-dimensional structure of the adenovirus major coat protein hexon. Science 232:1148-51.
29. Rux et al., 2003. Structural and phylogenetic analysis of adenovirus hexons by use of high-resolution x-ray crystallographic, molecular modeling, and sequence-based methods. J Virol 77:9553-66.
30. Schotsaert et al., 2009. Universal M2 ectodomain-based influenza A vaccines: preclinical and clinical developments. Expert Rev Vaccines 8:499-508.
31. Tatsis et al., 2007. Adenoviral vectors persist in vivo and maintain activated CD8+ T cells: implications for their use as vaccines. Blood 110:1916-23.
32. Thompson et al., 2006. Epidemiology of seasonal influenza: use of surveillance data and statistical models to estimate the burden of disease. J Infect Dis 194 Suppl 2:S82-91.
33. Ulmer et al., 1993. Heterologous protection against influenza by injection of DNA encoding a viral protein. Science 259:1745-9.
34. Veltrop-Duits et al., 2006. Human CD4+ T cells stimulated by conserved adenovirus 5 hexon peptides recognize cells infected with different species of human adenovirus. Eur J Immunol 36:2410-23.
35. Wang et al., 2010. Vaccination with a synthetic peptide from the influenza virus hemagglutinin provides protection against distinct viral subtypes. Proc Natl Acad Sci USA 107:18979-84.
36. Worgall et al., 2005. Protection against P. aeruginosa with an adenovirus vector containing an OprF epitope in the capsid. J Clin Invest 115:1281-9.
37. Xiang et al., 2002. Novel, chimpanzee serotype 68-based adenoviral vaccine carrier for induction of antibodies to a transgene product. J Virol 76:2667-75.
38. Xiang et al., 2006. Chimpanzee adenovirus antibodies in humans, sub-Saharan Africa. Emerg Infect Dis 12:1596-9.
39. Xue & Burnett, 2006. Capsid-like arrays in crystals of chimpanzee adenovirus hexon. J Struct Biol 154:217-21.
40. Yang et al., 1995. Cellular and humoral immune responses to viral antigens create barriers to lung-directed gene therapy with recombinant adenoviruses. J Virol 69:2004-15.
41. Zhao et al., 2010. An M2e-based multiple antigenic peptide vaccine protects mice from lethal challenge with divergent H5N1 influenza viruses. Virol J 7:9.
42. Zhou et al., 2010. A universal influenza A vaccine based on adenovirus expressing matrix-2 ectodomain and nucleoprotein protects mice from lethal challenge. Mol Ther 18:2182-9.
43. Zhou et al., 2010. An efficient method of directly cloning chimpanzee adenovirus as a vaccine vector. Nat Protoc 5:1775-85.

1. A modified adenovirus hexon protein comprising a first matrix protein ectodomain (M2e₁) from a first strain of influenza A virus inserted in a hypervariable region of the hexon protein.

2. The modified adenovirus hexon protein of claim 1 wherein the M2e₁ is inserted in hypervariable region 1.

3. The modified adenovirus hexon protein of claim 1 wherein the M2e₁ replaces three contiguous amino acids of hypervariable region 1.

4. The modified adenovirus hexon protein of claim 1 wherein the M2e₁ replaces amino acids 142-144 of the hexon protein shown in SEQ ID NO:6.

5. The modified adenovirus hexon protein of claim 1 wherein the M2e₁ is inserted in hypervariable region 4.

6. The modified adenovirus hexon protein of claim 1 wherein the M2e₁ is inserted between amino acids 253 and 254 of the hexon protein shown in SEQ ID NO:6.

7. The modified adenovirus hexon protein of claim 1, wherein the first strain of influenza virus is selected from the group consisting of an H1N1 strain, an H5N1 strain, an H7N2 strain, an H1N2 strain, an H2N2 strain, and an H3N2 strain.

8. A fusion protein comprising:
a second matrix protein ectodomain from a second strain of influenza A virus (M2e₂); and
a third matrix protein ectodomain from a third strain of influenza A virus (M2e₃); and
a fourth matrix protein ectodomain from a fourth strain of influenza A virus (M2e₄),
wherein at least two of the second, third, and fourth strains are different strains.

9. The fusion protein of claim 8, further comprising a nucleoprotein (NP) from a fifth strain of influenza A virus.

10. The fusion protein of claim 8, wherein components of the fusion protein are ordered, from N to C terminus, M2e₂-M2e₃-M2e₄-NP.

11. The fusion protein of claim 8, wherein the second strain of influenza virus is selected from the group consisting of an H1N1 strain, an H5N1 strain, an H7N2 strain, an H1N2 strain, an H2N2 strain, and an H3N2 strain.

12. A nucleic acid molecule selected from the group consisting of:
(a) a nucleic acid molecule encoding a modified adenovirus hexon protein comprising a first matrix protein ectodomain (M2e₁) from a first strain of influenza A virus inserted in a hypervariable region of the hexon protein; and
(b) a nucleic acid molecule encoding a fusion protein comprising:
a second matrix protein ectodomain from a second strain of influenza A virus (M2e₂); and
a third matrix protein ectodomain from a third strain of influenza A virus (M2e₃); and
a fourth matrix protein ectodomain form a fourth strain of influenza A virus (M2e₄),
wherein at least two of the second, third, and fourth strains are different strains.

13. An adenovirus comprising the modified adenovirus hexon protein of claim 1.

14. An adenovirus comprising the nucleic acid molecule of claim 12.

15. The adenovirus of claim 14, further comprising a modified adenovirus hexon protein comprising a first matrix protein ectodomain (M2e₁) from a first strain of influenza A virus inserted in a hypervariable region of the hexon protein.

16. An immunogenic composition comprising:
(1) an immunogenic component; and
(2) a pharmaceutically acceptable vehicle,
wherein the immunogenic component is selected from the group consisting of
(a) a modified adenovirus hexon protein comprising a first matrix protein ectodomain (M2e₁) from a first strain of influenza A virus inserted in a hypervariable region of the hexon protein;
(b) a fusion protein comprising: (1) a second matrix protein ectodomain from a second strain of influenza A virus (M2e₂); and (2) a third matrix protein ectodomain from a third strain of influenza A virus (M2e₃); and (3) a fourth matrix protein ectodomain from a fourth strain of influenza A virus (M2e₄), wherein at least two of the second, third, and fourth strains are different strains;
(c) a nucleic acid molecule encoding (a);
(d) a nucleic acid molecule encoding (b); and
(e) an adenovirus comprising (a).

17. A method of inducing an immune response to an influenza A virus, comprising a first administration of the composition of the immunogenic composition of claim 16 to an individual in need thereof.

18. The method of claim 17, further comprising a second administration of the composition.

19. The method of claim 17, wherein the first or second administration is selected from the group consisting of mucosal, oral, intramuscular, intravenous, and intraperitoneal administration.

20. The method of claim 17, wherein the immune response comprises antibody formation.

21. The method of claim 17, wherein the immune response comprises CD8⁺ T cell activation.

22-24. (canceled)


SEQUENCE LISTING (4 sequences)

SEQ ID NO: 1 - protein, 51 aa, Adenovirus
YNSLAPKGAPNTCQWTYKADGETATEKTYTYGNAPVQGINITKDGIQLGTD

SEQ ID NO: 2 - protein, 51 aa, Adenovirus
YNSLAPKGAPNSSQWEQAKTGNGGTMETHTYGVAPMGGENITKDGLQIGTD

SEQ ID NO: 3 - protein, 13 aa, Adenovirus
LTEVETPIRNEWG

SEQ ID NO: 4 - protein, 9 aa, Adenovirus
ASNENTETM